(19) United States
(12) Patent Application Publication
(10) Pub. No.: US 2018/0325879 A1
(43) Pub. Date: Nov. 15, 2018

(54) Antibacterial compositions

(71) Applicant: WOCKHARDT LIMITED, Chikalthana, Aurangabad (IN)

(72) Inventors: BHASKAR CHAUHAN, Sambhal-202412 (IN); MOHAMMED ASIF SHAIKH, Aurangabad-431001 (IN); RAJENDRA ARUN JOSHI, Aurangabad-431107 (IN); AJAYKUMAR HANDA, Aurangabad-431006 (IN); RAVINDRA DATTATRAYA YEOLE, AURANGABAD-431003 (IN); MAHESH VITHALBHAI PATEL, AURANGABAD-431003 (IN)

(73) Assignee: WOCKHARDT LIMITED, Chikalthana, Aurangabad (IN)

(21) Appl. No.: 15/547,511

(22) PCT Filed: Jul. 3, 2017
(86) PCT No.: PCT/IB2017/053998

§ 371 Date: Jul. 30, 2017

(30) Foreign Application Priority Data
Apr. 14, 2017 (IN) 201721013400

(51) Int. Cl. A61K 31/4375; A61K 9/08; A61K 47/02; A61K 47/18; A61K 9/00; A61K 31/444

(52) U.S. Cl. CPC A61K 31/4375; A61K 9/08; A61K 31/444; A61K 47/183; A61K 9/0019; A61K 47/02

(57) ABSTRACT

Pharmaceutical compositions comprising S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, and their use in treatment, control or prevention of bacterial infections is disclosed.

### RELATED PATENT APPLICATIONS

This application claims the priority to and benefit of Indian Provisional Patent Application No. 201721013400 filed on Apr. 14, 2017; the disclosures of which are incorporated herein by reference in its entirety as if fully rewritten herein.

### FIELD OF THE INVENTION

The invention relates to pharmaceutical compositions and their use in treatment, control or prevention of bacterial infections.

### BACKGROUND OF THE INVENTION

The infections caused by bacteria continue to be an area of significant concern globally. The emergence of bacterial resistance to known antibacterial agents is becoming a major challenge in treating bacterial infections. One way forward to treat bacterial infections, and especially those caused by resistant bacteria, is to develop new antibacterial agents that can overcome the bacterial resistance. Coates et al. (Br. J. Pharmacol. 2007; 152(8), 1147-1154) have reviewed approaches to developing new antibiotics. However, the development of new antibacterial agents is a challenging task. For example, Gwynn et al. (Annals of the New York Academy of Sciences, 2010, 1213: 5-19) have reviewed the challenges in the discovery of antibacterial agents. Several antibacterial agents and/or compositions have been described in the prior art. However, there remains a need for potent antibacterial agents and/or compositions for use in treatment, control and/or prevention of bacterial infections.

### SUMMARY OF THE INVENTION

Accordingly, there are provided pharmaceutical compositions and their use in treatment, control or prevention of bacterial infection in a subject.

In one general aspect, there is provided a pharmaceutical composition in the form of a solution, said solution comprising S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, said solution having pH within the range of about 8 to about 11.

In another general aspect, there is provided for use of a composition according to the invention in a method for treating, controlling or preventing bacterial infection.

In another general aspect, there is provided a method for treatment, control or prevention of a bacterial infection in a subject, said method comprising administering to said subject a composition according to the invention.

The details of one or more embodiments of the invention are set forth in the description below. Other features, objects and advantages of the invention will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, and additional applications of the principles of the invention as illustrated herein, which would occur to one of ordinary skills in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety.

The term “pharmaceutically acceptable salt” as used herein refers to one or more salts of a given compound which possesses the desired pharmacological activity of the free compound and which are neither biologically nor otherwise undesirable. In general, the “pharmaceutically acceptable salts” refer to and include those salts that are suitable for use in contact with the tissues of human and animals without undue toxicity, irritation, allergic response and the like, and are commensurate with a reasonable benefit/risk ratio. Pharmaceutically acceptable salts are well known in the art. For example, S. M. Berge, et al. (J. Pharmaceutical Sciences, 66: 1-19 (1977)), incorporated herein by reference in its entirety, describes various pharmaceutically acceptable salts in details.

The term “infection” or “bacterial infection” as used herein refers to and includes presence of bacteria, in or on a subject, which, if its growth were inhibited, would result in a benefit to the subject. As such, the term “infection” in addition to referring to the presence of bacteria also refers to normal flora, which is not desirable. The term “infection” includes infection caused by bacteria.

The term “treat”, “treating” or “treatment” as used herein refers to administering a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to administering treatment to a subject already suffering from infection. The terms “treat”, “treating” or “treatment” as used herein also refer to administering compositions, or one or more of pharmaceutically active ingredients discussed herein, with or without additional pharmaceutically active or inert ingredients, in order to: (i) reduce or eliminate either a bacterial infection, or one or more symptoms of the bacterial infection, or (ii) retard the progression of a bacterial infection, or of one or more symptoms of the bacterial infection, or (iii) reduce the severity of a bacterial infection, or of one or more symptoms of the bacterial infection, or (iv) suppress the clinical manifestation of a bacterial infection, or (v) suppress the manifestation of adverse symptoms of the bacterial infection.

The term “pharmaceutically effective amount” or “therapeutically effective amount” or “effective amount” as used herein refers to an amount, which has a therapeutic effect or is the amount required to produce a therapeutic effect in a subject. For example, a therapeutically or pharmaceutically effective amount of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). The pharmaceutically effective amount depends on several factors, including but not limited to, the microorganism (e.g. bacteria) involved, characteristics of the subject (for example height, weight, sex, age and medical history), severity of infection and the particular type of the antibacterial agent used. For prophylactic treatments, a therapeutically or prophylactically effective amount is that amount which would be effective in preventing a microbial (e.g. bacterial) infection. The compounds and/or pharmaceutical compositions according to the invention are used in amounts that are effective in providing the desired therapeutic effect or result.

The term “administration” or “administering” includes delivery of a composition, or one or more pharmaceutically active ingredients to a subject, including for example, by any appropriate methods, which serves to deliver the composition or its active ingredients or other pharmaceutically active ingredients to the site of the infection. The method of administration may vary depending on various factors, such as for example, the components of the pharmaceutical composition or the nature of the pharmaceutically active or inert ingredients, the site of the potential or actual infection, the microorganism involved, severity of the infection, age and physical condition of the subject and a like. Some non-limiting examples of ways to administer a composition or a pharmaceutically active ingredient to a subject according to this invention includes oral, intravenous, topical, intra-respiratory, intra-peritoneal, intra-muscular, parenteral, sublingual, transdermal, intranasal, aerosol, intra-ocular, intra-tracheal, intra-rectal, vaginal, gene gun, dermal patch, eye drop, ear drop or mouthwash. In case of a pharmaceutical composition comprising more than one ingredient (active or inert), one of way of administering such composition is by admixing the ingredients (e.g. in the form of a suitable unit dosage form such as tablet, capsule, solution, powder and a like) and then administering the dosage form. Alternatively, the ingredients may also be administered separately (simultaneously or one after the other) as long as these ingredients reach beneficial therapeutic levels such that the composition as a whole provides a synergistic and/or the desired effect.

The term “growth” as used herein refers to a growth of one or more microorganisms and includes reproduction or population expansion of the microorganism (e.g. bacteria). The term “growth” also includes maintenance of on-going metabolic processes of a microorganism (e.g. bacteria), including processes that keep the microorganism alive.

The term, “effectiveness” as used herein refers to ability of a treatment or a composition or one or more pharmaceutically active ingredients to produce a desired biological effect in a subject. For example, the term “antibacterial effectiveness” of a composition or an antibacterial agent refers to the ability of the composition or the antibacterial agent to treat or prevent the microbial (e.g. bacterial) infection in a subject.

The term “synergistic” or “synergy” as used herein refers to the interaction of two or more agents so that their combined effect is greater than their individual effects.

The term “pharmaceutically inert ingredient”,“inactive ingredient”, “carrier” or “excipient” refers to a compound or material used to facilitate administration of a compound, including for example, to increase the solubility of the compound. Typical, non-limiting examples of solid carriers include, starch, lactose, dicalcium phosphate, sucrose, and kaolin and so on. Typical, non-limiting examples of liquid carriers include sterile water, saline, buffers, non-ionic surfactants, and edible oils such as oil, peanut and sesame oils and so on. In addition, various adjuvants commonly used in the art may be included. These and other such compounds are described in the literature, for example, in the Merck Index (Merck & Company, Rahway, N.J.). Considerations for inclusion of various components in pharmaceutical compositions are described, for example, in Gilman et al. (Eds.) (1990); Goodman and Gilman's: The Pharmacological Basis of Therapeutics, 8th Ed., Pergamon Press., which is incorporated herein by reference in its entirety.

The term “subject” as used herein refers to a vertebrate or invertebrate, including a mammal. The term “subject” includes human, animal, a bird, a fish, or an amphibian. Typical, non-limiting examples of a “subject” includes humans, cats, dogs, horses, sheep, bovine cows, pigs, lambs, rats, mice and guinea pigs.

The term “parenteral administration” refers to and includes a route of administration that does not involve gastrointestinal tract directly. Typical, non-limiting examples of parenteral route of administration includes intravenous (into a vein), intra-arterial (into an artery), intraosseous infusion (into the bone marrow), intra-muscular, intracerebral, intrathecal, subcutaneous administration. In general, the parenteral administration is performed by injecting or infusing the composition or the active ingredient(s) directly into a subject without direct involvement of the gastrointestinal tract.

In one general aspect, there is provided a pharmaceutical composition in the form of a solution, said solution comprising S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, said solution having pH within the range of about 8 to about 11. In some embodiments, the solution has pH within the range of about 9 to about 10.5.

The compositions according to the invention are in the form of a solution and can be obtained by, for example, dissolving S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, in a pharmaceutically acceptable liquid diluent. Alternatively, the compositions according to the invention can also be obtained by dissolving any other compound capable of providing S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt when dissolved in a pharmaceutically acceptable diluent. Typical, non-limiting example of such other compounds include a various hydrate forms of S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, including the tetrahydrate form. Typical, non-limiting examples of such liquid diluent include water, sodium chloride solution, dextrose solution, saline solution and a like. In some embodiment, the composition according to the invention is obtained by dissolving S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt in water. In some other embodiments, the composition according to the invention is obtained by dissolving S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt tetrahydrate from in water.

In solution form, S-(−)-9-fluoro-6,7-dihydro-8-(4-hydroxypiperidin-1-yl)-5-methyl-1-oxo-1H,5H-benzo[i,j]quinolizine-2-carboxylic acid L-arginine may be present as such or in a dissociated form. Both these and any other forms are intended to be covered within the scope of the invention.

The compositions according to the invention are in the form of a solutionadapted to maintain pH within the range of about 8 to about 11. In some embodiments, the solution has pH within the range of about 9 to about 10.5. In general, the pH is maintained at a desired level by addition of suitable pH adjusting agent to the composition. In some embodiments, the pH is maintained at a desired level by addition of a buffer. In some embodiments, the pH is maintained at a desired level by addition of a sufficient quantity of L-arginine or a pharmaceutically acceptable salt thereof to the composition.

The amount of active and inactive ingredients in the composition according to the invention can vary depending on specific requirements. However, all active and inactive ingredients in the composition according to the invention are present in therapeutically effective amounts, such that the composition as a whole is therapeutically effective.

In some embodiments, S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt is present in the composition within the range of about 200 mg to about 2000 mg.

In some other embodiments, S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt is present in the composition within the range of about 400 mg to about 1500 mg.

In another general aspect, there are provided pharmaceutical compositions in the form of a solution, said solution having pH within the range of about 8 to about 11; said solution comprising: (a) S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, (b) L-arginine or a pharmaceutically acceptable salt thereof, and (c) sodium chloride.

In some embodiments, there are provided pharmaceutical compositions in the form of a solution, said solution having pH within the range of about 9 to about 10.5; said solution comprising: (a) S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, (b) L-arginine or a pharmaceutically acceptable salt thereof, and (c) sodium chloride.

In some embodiments, the pharmaceutical compositions according to the invention comprise L-arginine or a pharmaceutically acceptable salt thereof. In some embodiments, L-arginine or a pharmaceutically acceptable salt thereof is present in the composition within the range of about 200 mg to about 2000 mg. In some other embodiments, L-arginine or a pharmaceutically acceptable salt is present in the composition within the range of about 400 mg to about 1500 mg.

In some embodiments, the pharmaceutical compositions according to the invention comprise sodium chloride. In some other embodiments, sodium chloride is present in the composition within the range of about 200 mg to about 2000 mg. In some other embodiments, sodium chloride is present in the composition within the range of about 400 mg to about 1500 mg.

In some embodiments, there are provided pharmaceutical compositions in the form of a 100 ml solution, said solution having pH within the range of about 8 to about 11; said solution comprising: (a) about 200 to about 2000 mg of S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt, (b) about 200 to about 2000 mg of L-arginine or a pharmaceutically acceptable salt thereof, and (c) about 200 to about 2000 mg of sodium chloride.

In some embodiments, there are provided pharmaceutical compositions in the form of a 100 ml solution, said solution having pH within the range of about 9 to about 10.5; said solution comprising: (a) about 200 to about 2000 mg of S-0-9-fluoro-6,7-dihydro-8-(4-hydroxypiperidin-1-yl)-5-methyl-1-oxo-1H,5H-benzo[i,j] quinolizine-2-carboxylic acid L-arginine salt, (b) about 200 to about 2000 mg of L-arginine or a pharmaceutically acceptable salt thereof, and (c) about 200 to about 2000 mg of sodium chloride.

In some embodiments, the compositions according to the invention comprise about 2 mg/ml to about 20 mg/ml of S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt.In some other embodiments, the compositions according to the invention comprise about 2 mg/ml to about 20 mg/ml of L-arginine or a pharmaceutically acceptable salt thereof.In some other embodiments, the compositions according to the invention comprise about 2 mg/ml to about 20 mg/ml of sodium chloride.

In some embodiments, the compositions according to the invention haveosmolarity within the range of about 250 to about 350 mOsm/L.

The osmolarity may be adjusted to a desired level by addition of a suitable substance capable of modifying the osmolarity. In some embodiments, the osmolarityis maintained in the desired range by addition of sodium chloride.

The pharmaceutical compositions according to the invention may include one or more pharmaceutically acceptable carriers or excipients or a like. Typical, non-limiting examples of such carriers or excipients include mannitol, lactose, glucose, sucrose, emulsifying agents, solubilizing agents, pH buffering agents, stabilizing agents and a like.

The compositions according to the invention are stable over a wide range of storage conditions. In some embodiments, the compositions according to the invention comprise less than 5% w/w of total impurities. In some embodiments, the compositions according to the invention comprise less than 5% w/w of total impurities after storage at a temperature of about 40±2° C. and a relative humidity of about 75±5% for 3 months. In some embodiments, the compositions according to the invention comprise less than 5% w/w of total impurities after storage at a temperature of about 25±2° C. and a relative humidity of about 60±5% for 24 months.

In some embodiments, the compositions according to the invention comprise one or more of the following:

(i) less than 0.25% w/w of 9-fluoro-8-hydroxy-5-methyl-6,7-dihydro-1-oxo-1H,5H-benzo[i,j]quinolizine-2-carboxylic acid;

(ii) less than 3% w/w of 9-fluoro-6,7-dihydro-8-amino-5-methyl-1-oxo -1H,5H-benzo[i,j] quinolizine-2-carboxylic acid;

(iii) less than 0.25% w/w of 8-fluoro-9-(4-hydroxy-piperidin-1-yl)-5-methyl-6,7-dihydro-1-oxo-1H,5H-benzo[i,j]quinolizine-2-carboxylic acid;

(iv) less than 0.25% w/w of 8-(4-hydroxy-1-piperidinyl)-5-methyl-6,7-dihydro-1-oxo-1H, 5H-benzo[i,j]quinolizine-2-carboxylic acid;

(v) less than 0.25% w/w of 8,9-difluoro-5-methyl-6,7-dihydro-1-oxo-1H,5H-benzo[i,j] quinolizine-2-carboxylic acid; or

(vi) less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid.

In some other embodiments, the compositions according to the invention comprise one or more of the following after storage at a temperature of about 40±2° C. and a relative humidity of about 75±5% for 3 months:

(i) less than 0.25% w/w of 9-fluoro-8-hydroxy-5-methyl-6,7-dihydro-1-oxo-1H,5H-benzo[i,j]quinolizine-2-carboxylic acid;

(ii) less than 3% w/w of 9-fluoro-6,7-dihydro-8-amino-5-methyl-1-oxo -1H,5H-benzo[i,j] quinolizine-2-carboxylic acid;

(iii) less than 0.25% w/w of 8-fluoro-9-(4-hydroxy-piperidin-1-yl)-5-methyl-6,7-dihydro-1-oxo-1H,5H-benzo[i,j]quinolizine-2-carboxylic acid;

(iv) less than 0.25% w/w of 8-(4-hydroxy-1-piperidinyl)-5-methyl-6,7-dihydro-1-oxo-1H, 5H-benzo[i,j]quinolizine-2-carboxylic acid;

(v) less than 0.25% w/w of 8,9-difluoro-5-methyl-6,7-dihydro-1-oxo-1H,5H-benzo[i,j] quinolizine-2-carboxylic acid; or

(vi) less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid.

In some other embodiments, the compositions according to the invention comprise one or more of the following after storage at a temperature of about 25±2° C. and a relative humidity of about 60±5% for 24 months:

(i) less than 0.25% w/w of 9-fluoro-8-hydroxy-5-methyl-6,7-dihydro-1-oxo-1H,5H-benzo[i,j]quinolizine-2-carboxylic acid;

(ii) less than 3% w/w of 9-fluoro-6,7-dihydro-8-amino-5-methyl-1-oxo -1H,5H-benzo[i,j] quinolizine-2-carboxylic acid;

(iii) less than 0.25% w/w of 8-fluoro-9-(4-hydroxy-piperidin-1-yl)-5-methyl-6,7-dihydro-1-oxo-1H,5H-benzo[i,j]quinolizine-2-carboxylic acid;

(iv) less than 0.25% w/w of 8-(4-hydroxy-1-piperidinyl)-5-methyl-6,7-dihydro-1-oxo-1H, 5H-benzo[i,j]quinolizine-2-carboxylic acid;

(v) less than 0.25% w/w of 8,9-difluoro-5-methyl-6,7-dihydro-1-oxo-1H,5H-benzo[i,j] quinolizine-2-carboxylic acid; or

(vi) less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid.

In some embodiments, the pharmaceutical compositions according to the invention are present in the form of a solution that can be reconstituted by addition of a compatible reconstitution diluent prior to administration. In some embodiments, the pharmaceutical compositions according to the invention are present in the form of ready-to-use solution for parenteral administration. In some embodiments, the pharmaceutical compositions according to the invention are present in the form of a solution as a unit dose contained in a bottle, vial or bag prior to parenteral administration. In some other embodiments, the pharmaceutical compositions according to the invention are in the form of a frozen composition that can be diluted with a compatible reconstitution diluent prior to administration. A wide variety of reconstitution diluents can be used. Typical, non-limiting example of reconstitution diluent includes water for injection, 0.9% sodium chloride solution, 5% dextrose solution, normal saline solution and a like.

In another general aspect, the compositions according to the invention are useful in treatment, control or prevention of a bacterial infection. In some embodiments, the compositions according to the invention are used in the preparation of a medicament for treatment,control, or prevention of bacterial infection.

In another general aspect, there is provided a method for treatment, control and/or prevention of bacterial infection in a subject, said method comprising administering to said subject a composition according to the invention. In some embodiments, the composition is administered parenterally.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Various ingredients used in the following example are summarized below:
Compound A:
S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt.
Compound B:
9-fluoro-8-hydroxy-5-methyl-6,7-dihydro-1-oxo-1H,5H-benzo [i,j]quinolizine-2-carboxylic acid.
Compound C:
9-fluoro-6,7-dihydro-8-amino-5-methyl-1-oxo -1H,5H-benzo[i,j] quinolizine-2-carboxylic acid.
Compound D:
8-fluoro-9-(4-hydroxy-piperidin-1-yl)-5-methyl-6,7-dihydro-1-oxo-1H,5H-benzo[i,j]quinolizine-2-carboxylic acid.
Compound E:
8-(4-hydroxy-1-piperidinyl)-5-methyl-6,7-dihydro-1-oxo-1H, 5H-benzo[i,j]quinolizine-2-carboxylic acid.
Compound F:
8,9-difluoro-5-methyl-6,7-dihydro-1-oxo-1H,5H-benzo[i,j] quinolizine-2-carboxylic acid.
Compound G:
R-(+)-9-fluoro-8-(4-hydroxy-piperidin-1-yl)-5 -methyl-6,7-dihydro-1-oxo-1H,5H-benzo[i,j]quinolizine-2-carboxylic acid.

A wide variety of compositions comprising various ingredients (active as well as inactive ingredients) were prepared. The amount of such ingredients (active as well as inactive) in these compositions was within the ranges as described. The pH and osmolarity of these compositions varied over a wide range, including those described herein. Details of a few examples are given in Table 1, below:

TABLE 1

| Ingredients | Composition 1 | | Composition 2 | |
|---|---|---|---|---|
| Compound A | 1186.7 | mg | 890 | mg |
| L-Arginine | 1333 | mg | 1000 | mg |
| Sodium Chloride | 580 | mg | 670 | mg |
| Nitrogen | q.s. to purge | | q.s. to purge | |
| Water for Injection | q.s. to 100 | ml | q.s. to 100 | ml |

In general, L-Arginine, Compound A, and sodium chloride were sequentially added to appropriate amount of water (water for injection having dissolved oxygen content less than 2 ppm). The bulk solution was filtered using 1.2 μPP filter and 0.2 μPES filters. The filtered solution was packed into bottle and sterilized. The stability of the prepared compositions was determined at appropriate conditions and the results are given in Table 2 (for Composition 1) and Table 3 (for Composition 2).

TABLE 2

| | | Values | | |
|---|---|---|---|---|
| | | | After storage at 40 ± 2° C. | After storage at 25 ± 2° C. |
| | | | and 75 ± 5% relative | and 60 ± 5% relative |
| Sr. | Test | Initial | humidity for 3 months | humidity for 24 months |
| 1. | Assay of Compound A | 98.7 | 100.4 | 100.1 |
| 2. | Assay of NaCl | 100.7 | 100.7 | 99.3 |
| 3. | Related substances as determined by HPLC | | | |
| 4. | Compound B | 0.000 | 0.000 | 0.000 |
| 5. | Compound C | 0.138 | 0.381 | 0.809 |
| 6. | Compound D | 0.000 | 0.000 | 0.000 |
| 7. | Compound E | 0.019 | 0.053 | 0.023 |
| 8. | Compound F | 0.000 | 0.000 | 0.000 |
| 9. | Compound G | 0.000 | 0.000 | 0.000 |
| 10. | Total impurities | 0.157 | 0.503 | 0.935 |
| 11. | pH of the composition | 9.67 | 9.50 | 9.73 |
| 12. | Osmolarity (mOsm/L) | 291 | 291 | 296 |

TABLE 3

| | | Values | | |
|---|---|---|---|---|
| | | | After storage at 40 ± 2° C. | After storage at 25 ± 2° C. |
| | | | and 75 ± 5% relative | and 60 ± 5% relative |
| Sr. | Test | Initial | humidity for 3 months | humidity for 24 months |
| 1. | pH of the composition | 9.42 | 9.65 | 9.58 |
| 2. | Assay of Compound A | 102.8 | 101.5 | 102.7 |
| 3. | Assay of NaCl | 100.98 | 102.1 | 100.0 |
| 4. | Related substances (by HPLC) | | | |
| 5. | Compound B | 0.000 | 0.000 | 0.005 |
| 6. | Compound C | 0.131 | 0.508 | 0.608 |
| 7. | Compound D | 0.000 | 0.000 | 0.000 |
| 8. | Compound E | 0.049 | 0.049 | 0.057 |
| 9. | Compound F | 0.000 | 0.000 | 0.000 |
| 10. | Compound G | 0.000 | 0.000 | 0.000 |
| 11. | Total impurities | 0.180 | 0.624 | 0.777 |

1. A pharmaceutical composition in the form of a solution, said solution comprising S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, said solution having pH within the range of about 8 to about 11.

2. The composition according to claim 1, wherein said solution has pH within the range of about 9 to about 10.5.

3. The composition according to claim 1 or 2, wherein the pH is maintained within the range by addition of a sufficient quantity of L-arginine or a pharmaceutically acceptable salt thereof.

4. The composition according to claim 1 or 2, wherein the pH is maintained within the range by addition of a sufficient quantity of one or more buffers.

5. The composition according to any one of the claims 1 to4, wherein the solution has osmolarity within the range of about 250 to about 350 mOsm/L.

6. The composition according to claim 5, wherein the osmolarityis maintained within the range by addition of sodium chloride.

7. The pharmaceutical composition according to any one of the claims 1 to 6, wherein S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt is present in the composition within the range of about 200 mg to about 2000 mg.

8. The pharmaceutical composition according to any one of the claims 1 to 6, wherein S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt is present in the composition within the range of about 400 mg to about 1500 mg.

9. A pharmaceutical composition in the form of a solution, said solution having pH within the range of about 8 to about 11; said solution comprising: (a) S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, (b) L-arginine or a pharmaceutically acceptable salt thereof, and (c) sodium chloride.

10. The composition according to claim 9, wherein said solution has pH within the range of about 9 to about 10.5.

11. The pharmaceutical composition according to any one of the claims 9 to 10, wherein S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt is present in the composition within the range of about 200 mg to about 2000 mg.

12. The pharmaceutical composition according to any one of the claims 9 to 10, wherein S-(−)-9-fluoro-6,7-dihydro-8-(4-hydroxypiperidin-1-yl)-5-methyl-1-oxo-1H,5H-benzo[i,j] quinolizine-2-carboxylic acid L-arginine salt is present in the composition within the range of about 400 mg to about 1500 mg.

13. The pharmaceutical composition according to any one of the 9 to 10, wherein L-arginine or a pharmaceutically acceptable salt is present in the composition within the range of about 200 mg to about 2000 mg.

14. The pharmaceutical composition according to any one of the claims 9 to 10, wherein L-arginine or a pharmaceutically acceptable salt is present in the composition within the range of about 400 mg to about 1500 mg.

15. The pharmaceutical composition according to any one of the claims 9 to 10, wherein sodium chloride is present in the composition within the range of about 200 mg to about 2000 mg.

16. The pharmaceutical composition according to any one of the claims 9 to 10, wherein sodium chloride is present in the composition within the range of about 400 mg to about 1500 mg.

17. A pharmaceutical composition in the form of a 100 ml solution, said solution having pH within the range of about 8 to about 11; said solution comprising: (a) about 200 mg to about 2000 mg of S-(−)-9-fluoro-6,7-dihydro-8-(4-hydroxypiperidin-1-yl)-5-methyl-1-oxo-1H,5H-benzo[i,j]quinolizine-2-carboxylic acid L-arginine salt, (b) about 200 mg to about 2000 mg of L-arginine or a pharmaceutically acceptable salt thereof, and (c) about 200 to about 2000 mg of sodium chloride.

18. The composition according to claim 18, wherein said solution has pH within the range of about 9 to about 10.5

19. The pharmaceutical composition according to any one of the claims 9 to 18, wherein said solution has osmolarity within the range of about 250 to about 350 mOsm/L.

20. The composition according to any one of the claims 1 to 19, said composition comprising less than 5% w/w of total impurities.

21. The composition according to any one of the claims 1 to 19, said composition comprising less than 5% w/w of total impurities after storage at a temperature of about 40±2° C. and a relative humidity of about 75±5% for 3 months.

22. The composition according to any one of the claims 1 to 19, said composition comprising less than 5% w/w of total impurities after storage at a temperature of about 25 ±2° C. and a relative humidity of about 60 ±5% for 24 months.

23. The composition according to any one of the claims 1 to 19, said composition comprising one or more of the following:
(i) less than 0.25% w/w of 9-fluoro-8-hydroxy-5-methyl-6,7-dihydro-1-oxo-1H,5H-benzo[i,j]quinolizine-2-carboxylic acid;
(ii) less than 3% w/w of 9-fluoro-6,7-dihydro-8-amino-5-methyl-1-oxo -1H,5H-benzo[i,j]quinolizine-2-carboxylic acid;
(iii) less than 0.25% w/w of 8-fluoro-9-(4-hydroxy-piperidin-1-yl)-5-methyl-6,7-dihydro-1-oxo-1H,5H-benzo[i,j]quinolizine-2-carboxylic acid;
(iv) less than 0.25% w/w of 8-(4-hydroxy-1-piperidinyl)-5-methyl-6,7-dihydro-1-oxo-1H, 5H-benzo[i,j]quinolizine-2-carboxylic acid;
(v) less than 0.25% w/w of 8,9-difluoro-5-methyl-6,7-dihydro-1-oxo-1H,5H-benzo[i,j] quinolizine-2-carboxylic acid; or
(vi) less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid.

24. The composition according to any one of the claims 1 to 19, said composition comprising one or more of the following after storage at a temperature of about 40±2° C. and a relative humidity of about 75±5% for 3 months:
(i) less than 0.25% w/w of 9-fluoro-8-hydroxy-5-methyl-6,7-dihydro-1-oxo-1H,5H-benzo[i,j]quinolizine-2-carboxylic acid;
(ii) less than 3% w/w of 9-fluoro-6,7-dihydro-8-amino-5-methyl-1-oxo-1H,5H-benzo[i,j] quinolizine-2-carboxylic acid;
(iii) less than 0.25% w/w of 8-fluoro-9-(4-hydroxy-piperidin-1-yl)-5-methyl-6,7-dihydro-1-oxo-1H,5H-benzo[i,j]quinolizine-2-carboxylic acid;
(iv) less than 0.25% w/w of 8-(4-hydroxy-1-piperidinyl)-5-methyl-6,7-dihydro-1-oxo-1H, 5H-benzo[i,j]quinolizine-2-carboxylic acid;
(v) less than 0.25% w/w of 8,9-difluoro-5-methyl-6,7-dihydro-1-oxo-1H,5H-benzo[i,j] quinolizine-2-carboxylic acid; or
(vi) less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid.

25. The composition according to any one of the claims 1 to 19, said composition comprising one or more of the following after storage at a temperature of about 25±2° C. and a relative humidity of about 60±5% for 24 months:
(i) less than 0.25% w/w of 9-fluoro-8-hydroxy-5-methyl-6,7-dihydro-1-oxo-1H,5H-benzo[i,j]quinolizine-2-carboxylic acid;
(ii) less than 3% w/w of 9-fluoro-6,7-dihydro-8-amino-5-methyl-1-oxo -1H,5H-benzo[i,j] quinolizine-2-carboxylic acid;
(iii) less than 0.25% w/w of 8-fluoro-9-(4-hydroxy-piperidin-1-yl)-5-methyl-6,7-dihydro-1-oxo-1H,5H-benzo[i,j]quinolizine-2-carboxylic acid;
(iv) less than 0.25% w/w of 8-(4-hydroxy-1-piperidinyl)-5-methyl-6,7-dihydro-1-oxo-1H, 5H-benzo[i,j]quinolizine-2-carboxylic acid;
(v) less than 0.25% w/w of 8,9-difluoro-5-methyl-6,7-dihydro-1-oxo-1H,5H-benzo[i,j] quinolizine-2-carboxylic acid; or
(vi) less than 0.5% w/w of R-(+)-9-fluoro-8-(4-hydroxy-piperidin-1-yl)-5-methyl-6,7-dihydro-1-oxo-1H,5H-benzo[i,j]quinolizine-2-carboxylic acid.

26. Use of a composition according to any one of the claims 1 to 25, in a method for treating, controlling or preventing bacterial infection.

27. Use of a composition according to any one of the claims 1 to 25, in the preparation of a medicament for treatment, control or prevention of bacterial infection.

28. A method for treatment, control or prevention of a bacterial infection in a subject, said method comprising administering to said subject the composition according to any one of the claims 1 to 25.

29. The method according to claim 28, wherein the composition is administered parenterally.
